(19) United States
(12) Patent Application Publication
(10) Pub. No.: US 2021/0298581 A1
(43) Pub. Date: Sep. 30, 2021

(54) ADJUSTABLE-SHAPE SNARE FOR ENDOSCOPIC PROCEDURES

(71) Applicant: Endovision Foundation, San Francisco, CA (US)

(72) Inventors: Kenneth F. Binmoeller, Rancho Santa Fe, CA (US); John P. Lunsford, San Carlos, CA (US)

(73) Assignee: Endovision Foundation, San Francisco, CA (US)

(21) Appl. No.: 17/346,180

(22) Filed: Jun. 11, 2021

Related U.S. Application Data
(63) Continuation-in-part of application No. 16/775,219, filed on Jan. 28, 2020.
Application No. 16/775,219 is a continuation of application No. 14/777,393, filed on Sep. 15, 2015, now Pat. No. 10,542,871.
(60) Provisional application No. 61/801,427, filed on Mar. 15, 2013.
(60) Provisional application No. 61/786,520, filed on Mar. 15, 2013.

(51) Int. Cl. A61B 1/00; A61B 1/012; A61B 1/04; A61B 1/31; A61B 17/32; A61B 17/3205

(52) U.S. Cl. CPC A61B 1/00137; A61B 1/0008; A61B 1/00101; A61B 1/00131; A61B 17/32056; A61B 1/00163; A61B 1/04; A61B 1/31; A61B 17/320016; A61B 1/012

(57) ABSTRACT

An endoscopic snare tool with a loop wire positioned substantially within a catheter shaft and capable of deploying from and retracting into a distal end thereof at a consistent shape and angle. The shaft may include a guide tip with bisecting bores which guides the loop wire as it deploys or retracts from a lateral position of the shaft so that a consistent shape and angle are maintained. The loop wire may be formed from a composite design or a specially-treated monofilament to further maintain the shape of the loop during reduction or expansion and may further include a torsion spring positioned at a tip of the loop wire to additionally maintain the circular shape of the loop and aid in excision of tissue.

### CROSS-REFERENCE TO RELATED APPLICATIONS

This application is a continuation-in-part of U.S. application Ser. No. 16/775,219, filed Jan. 28, 2020, which is a continuation of U.S. application Ser. No. 14/777,393, filed Sep. 15, 2015, now U.S. patent Ser. No. 10/542,871, issued Jan. 28, 2020, which is a U.S. national phase filing of International Patent Application No. PCT/US2014/30568, entitled SYSTEMS, METHODS AND DEVICES FOR PERFORMING WATER AIDED ENDOSCOPY, which claims benefit of priority to U.S. Provisional Patent Application No. 61/801,427, filed Mar. 15, 2013, and U.S. Provisional Application No. 61/786,520, filed Mar. 15, 2013, the contents of the above applications are incorporated herein by reference in their entireties for all purposes.

### BACKGROUND

### Field of the Invention

The embodiments described herein are related to devices and methods for performing endoscopic procedures, and more particularly to a snare for performing excisions and markings.

### Related Art

Endoscopy is a minimally invasive medical procedure where an endoscope is inserted into a body cavity in order to view the interior of the body cavity. If the body cavity has an existing orifice, such as the colon or esophagus, the endoscope can be inserted through the orifice. A gas—either air or carbon dioxide—or water is introduced into the cavity to distend the body cavity for better viewing by the endoscope. Although endoscopy may be used simply to view the body cavity for diagnostic purposes, one or more tools may be inserted into the body cavity at the same time in order to perform an interventional diagnostic or therapeutic procedure.

One of the common tools used during an endoscopic procedure is a snare, which has a hollow catheter shaft which retains a pair of wires formed into a loop at a distal end of the tube which is then inserted into a body cavity. The loop can be expanded from the distal end of the tube into an oval shape, which can then encompass a polyp or other area of tissue that needs to be removed. The loop is then retracted until the edges of the loop press upon the base of the polyp and excise it from the surrounding tissue. Thus, these snares are often termed “polypectomy snares” due to their primary function in removing polyps during an endoscopy procedure. Snares can be connected to a high frequency generator to produce an electric charge to heat the snare to aid in excising the polyp and to coagulate the excised tissue area in a procedure known as “hot snare polypectomy”. Snare excision that does not require diathermy is termed “cold snare polypectomy.”

The primary limitation of the snare is the shape and movement of the loop. Due to the structure of the wire and the sheath housing the wire, the loop sides collapse together and take a generally oval-shape, as shown by the image of a polypectomy snare 10 in FIG. 1. The vertical oval snare 10 has a sheath 6 which substantially retains a wire loop 2 that is then extended from the sheath 6 into a deployed position where it takes on an oval shape. It may also include a crimped tip 4 which is used to help the loop return to its reduced shape entirely within the sheath 6. The vertical oval shape is problematic in that the shape may not allow the widest opening of the snare to completely encompass the polyp perimeter, and the oval shape narrows to a linear shape as the snare loop is closed. Furthermore, the wire is often thin and can be difficult to control from deforming and rotating while it is being reduced into the sheath. It often requires careful precision in placing the loop around the object, and a slow and methodical reduction of the loop so that it stays in place around the object until it is close enough to excise.

Therefore, there is a need for snares which address and improve upon these limitations.

### SUMMARY

Embodiments described herein include an endoscopic snare tool with a loop wire positioned substantially within a catheter shaft and capable of deploying from and retracting into a distal end thereof at a desired shape and angle. The shaft may include a guide tip with bisecting bores which guides the loop wire as it deploys or retracts from a lateral position of the shaft so that a consistent shape and angle with respect to an ensnared object are maintained. The loop wire may be formed from a composite braided design or a specially-treated monofilament to further maintain the shape of the loop during reduction or expansion and may further include a torsion spring positioned at a tip of the loop wire to additionally maintain the circular shape of the loop and aid in excision of tissue.

In one embodiment, an endoscopic snare tool comprises a hollow outer sheath with a proximal end and a distal end, wherein the distal end is configured to pass through a channel in an endoscope; an adjustable wire substantially disposed within the hollow outer tube, wherein the adjustable wire forms a circular loop shape at the distal end of the hollow outer tube; and wherein the circular loop shape of the adjustable wire is maintained as a diameter of the loop shape is expanded or reduced.

In another embodiment, a method of ensnaring an object with an endoscopic snare tool comprises the steps of: inserting the snare tool through a channel in an endoscope and into a body cavity; expanding an adjustable loop wire disposed within the snare tool into the body cavity in a substantially circular shape; positioning the adjustable loop around the object; reducing the adjustable loop to ensnare the object while maintaining the substantially circular shape; and withdrawing the snare tool and reduced adjustable loop wire from the channel in the endoscope.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is an image of a polypectomy snare with a wire loop in an expanded position from a sheath, as known in the art.

FIG. 2 is a perspective view illustration of an adjustable-shape snare showing the expanded circular wire loop with an outside-facing torsion spring, according to an embodiment of the invention;

FIG. 3 is a perspective view illustration of the adjustable-shape snare with the circular wire loop in the expanded position with an inside-facing torsion spring, according to an embodiment of the invention;

FIG. 4A is an illustration of a guide tip located on a distal end of the sheath for retaining the wire loop, according to one embodiment of the invention;

FIG. 4B is a cross-section illustration of the guide tip illustrating converging bores for retaining the wire loop, according to an embodiment of the invention;

FIG. 4C is a perspective view illustration of a wire loop with a cutting edge, according to one embodiment of the invention;

FIG. 4D is a front-view illustration of the wire guide with a cutting edge, according to one embodiment of the invention;

FIG. 4E is a cross-section view illustration of the interface between the wire loop cutting edge and the wire guide cutting edge, according to one embodiment of the invention;

FIG. 5A-5C are cross-sectional view illustrations of composite wire designs for forming the wire loop, as known in the art;

FIG. 6 is a perspective view image of a braided tube surrounding the wire loop, according to one embodiment of the invention;

FIG. 7 is a perspective view image of a process of crimping the wire loop, according to one embodiment of the invention;

FIG. 8A is an illustration of the adjustable-shape snare at a first diameter in a fully-expanded configuration, according to one embodiment of the invention;

FIG. 8B is an illustration of the adjustable-shape snare at a second diameter in a partially-expanded configuration, according to one embodiment of the invention;

FIG. 8C is an illustration of the adjustable-shape snare at a third diameter in a fully-reduced configuration, according to one embodiment of the invention;

FIG. 8D is a cross-section illustration of a swivel mechanism for the adjustable-shape snare (with internal elements shown in dashed lines), according to one embodiment of the invention;

FIG. 9 is a flow diagram illustrating an example process for using a snare with a consistent-shape wire loop, according to an embodiment of the invention.

### DETAILED DESCRIPTION

Certain embodiments disclosed herein provide an endoscopic snare tool with a loop wire positioned substantially within a catheter shaft and capable of expanding from and retracting into a distal end thereof at a consistent shape and angle with respect to an object to be ensnared therewith. The shaft may include a guide tip with bisecting bores which guides the loop wire as it deploys or retracts from a lateral position of the shaft so that a consistent shape and angle are maintained. The loop wire may be formed from a composite design or a specially-treated monofilament to further maintain the shape of the loop during reduction or expansion, and may further include a torsion spring positioned at a tip of the loop wire to additionally maintain the shape of the loop and aid in excision of tissue.

As will be described further below, the snare tool significantly improves upon existing devices by providing a wire loop which may retain the same shape as the loop is deployed from the distal tip and, more importantly, as it is reduced to encompass and excise tissue such as during a polypectomy. The wire materials, structure, shape and treatments, along with the use of additional structural changes like the torsion spring at the distal tip, all further aid the wire in retaining a specific shape, which allows a user performing an endoscopic procedure with the snare tool to more easily achieve en bloc resection with disease-free tissue margins (RO resection). The consistent shape of the loop and angle with respect to the target tissue keeps the loop securely positioned around the perimeter of the target tissue, as opposed to a conventional vertical-oval snare that becomes narrower in diameter as it is reduced, losing its ability to evenly cinch around target tissue. The wire may also be configured to form a horizontal-oval shape as the loop is reduced in order to capture and excise common polyps and other tissues which extend horizontally.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

I. Snare Tool with Torsion Spring

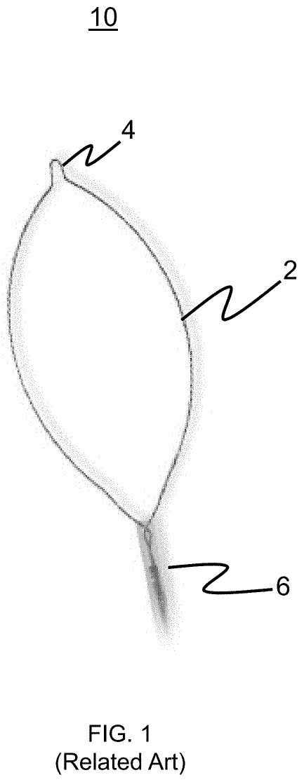

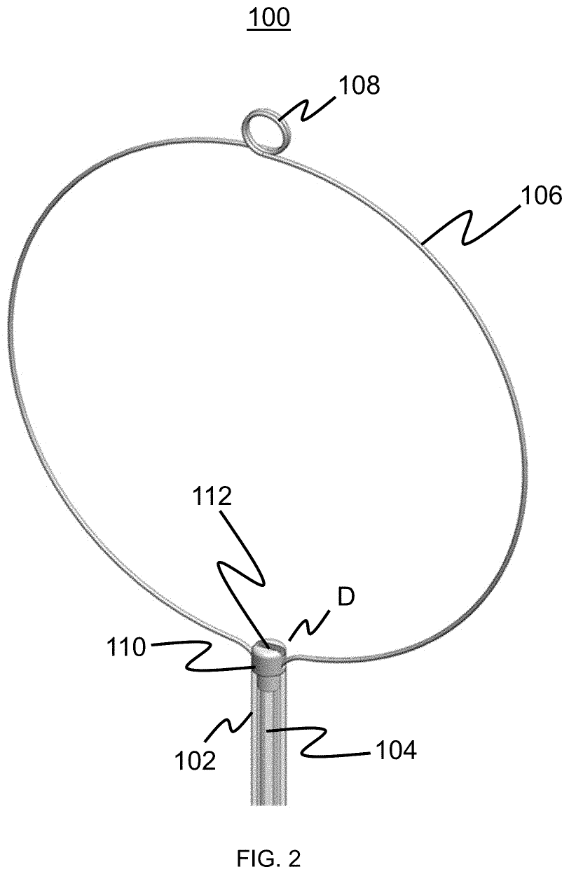

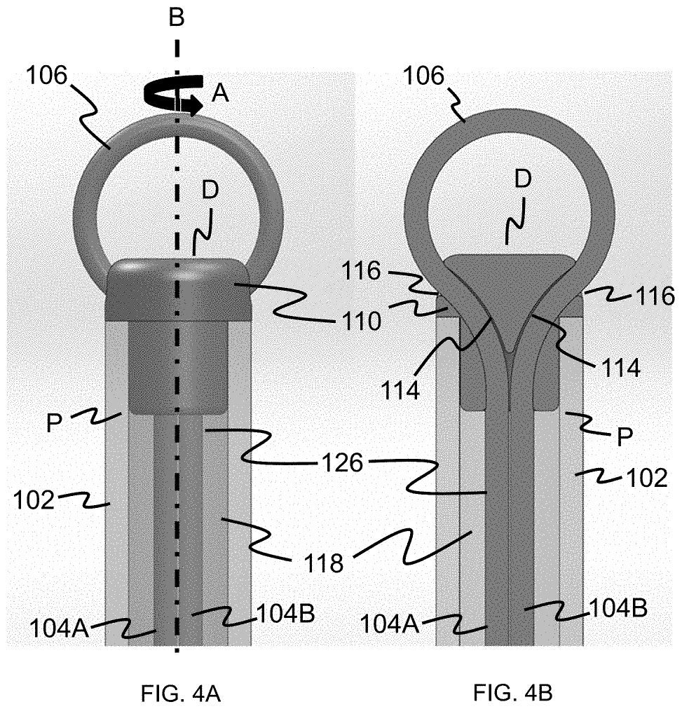

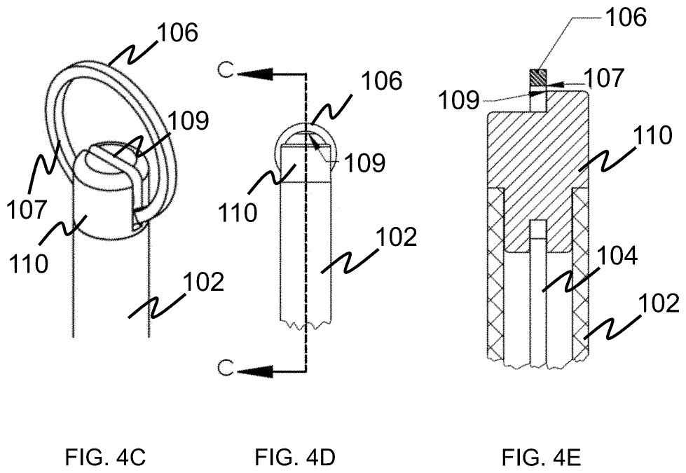

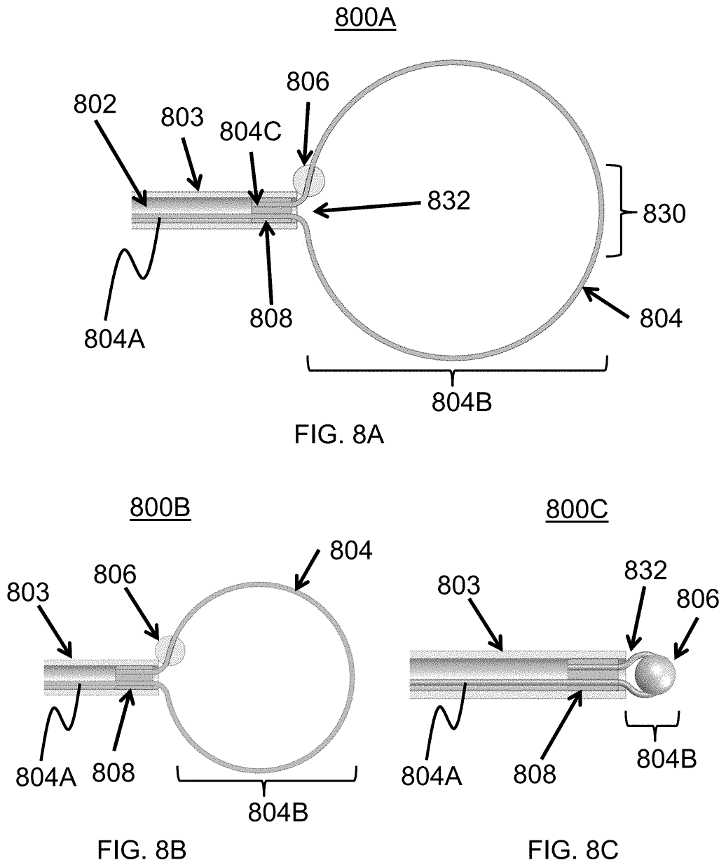

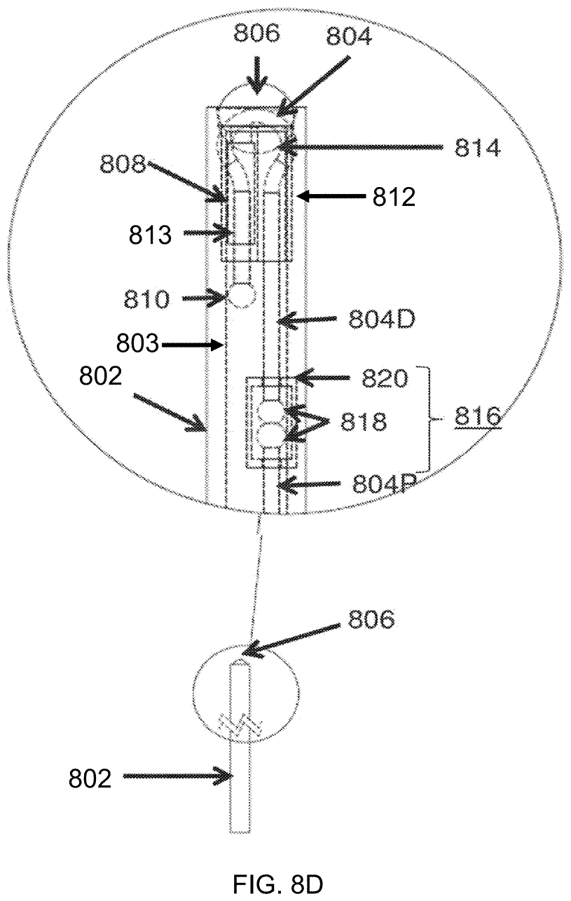

In one embodiment of an endoscopic snare tool illustrated in FIG. 2, the snare tool 100 includes a hollow tube or catheter shaft 102 which, in this deployed position, retains part of a loop wire 104 while allowing a loop portion 106 of the loop wire 104 to deploy into a circular shape at a distal end D of the tube 102. In contrast to the oval shape of the related art device illustrated in FIG. 1, the loop portion 106 is circular and is capable of maintaining this circular shape as the loop portion 106 is deployed from a retracted position (as shown in FIGS. 4A, 4B and 8C, below) to an expanded position shown here. One feature that will maintain the circular shape of the loop portion 106 is a torsion spring 108 which may be disposed at a point on a circumference of the loop wire, in this case at a distal tip of the loop such that it is directly across from the distal end of the shaft.

The torsion spring 108 absorbs the force being applied to the wire along its lateral sides as it expands, allowing the lateral sides to extend further in the lateral directions to maintain the circular shape, in contrast to the related art devices which have largely straight lateral sides that creates the oval shape shown in FIG. 1. As the loop is reduced, the spring 108 will unwind and increase in size, increasing outward force on the lateral sides to keep the loop in the circular shape.

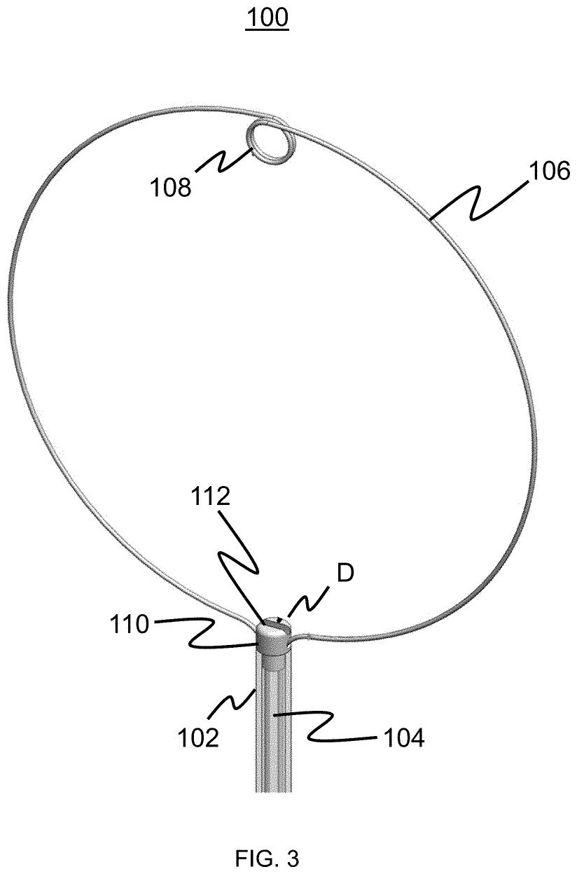

In the embodiment illustrated in FIG. 3, the torsion spring 108 may be disposed on an inner circumference of the loop portion 106. In this embodiment, the torsion spring 108 will wind up and reduce in size as the loop portion 106 is reduced, also increasing outward force on the lateral sides of the loop portion 106 to maintain the circular shape of the loop. Having the torsion spring on the inner circumference may also provide higher resistance to plastic deformation (where the wire is permanently bent) during the reduction of the loop portion 106 than the outward-facing torsion spring in FIG. 2.

The torsion spring 108 may also function as a cleat or anchor to engage adjacent soft tissue and maintain the loop portion 106 in place around a polyp or other tissue to be removed and prevent the loop portion 106 from slipping out of place as it is reduced to surround the polyp.

In one embodiment illustrated in both FIG. 2 and FIG. 3, the distal end D of the tube 102 may include a guide tip 110 with a slot 112 which can partially retain the torsion spring in the outer or inner configurations and will aid in shearing tissue during a cold-snaring procedure. If a portion of the loop is electrified and heated for hot snaring or monopolar marking (such as the process of fulguration of the margins after a polyp has been removed), the presence of the torsion spring 108 within the slot 112 will also increase the surface area that can be used to mark an area of tissue with electrocautery and can also act as a focal point for greater accuracy in marking.

II. Guide Tip

FIG. 4A illustrates one embodiment of an endoscopic snare tool in a reduced or retracted position after the loop portion 106 has been reduced back into the shaft 102. The retracted wire 104A is deformed into a retracted position 126 in a substantially linear shape parallel to a corresponding opposing internal end of the wire 104B within an internal housing 118. Although not illustrated here, a proximal end of the snare tool may have a handle in movable connection with one or both portions of the retracted wire 104A and internal wire 104B that allows an operator to manipulate the wire to deploy or retract the loop portion 106 and even rotate the loop portion 106 in the direction of Arrow A with respect to a lengthwise axis B of the shaft. For example, loop 106 in FIG. 1 may be generally reduced by simultaneous retraction of both ends of the wire 104A and 104B, while in other embodiments (such as that shown in FIG. 8A) the loop is reduced by retracting just one end of the wire 804. Furthermore, both ends 104A, 104B of the wire could be retracted at different rates to avoid damaging one section of wire through repeated acute bends—using a gearing mechanism in a handle—to alter a bending center of the loop. The entire shaft may also be rotated to easily rotate the loop portion in an identical fashion.

FIG. 4B is a cross-sectional view of the tool in FIG. 4A, illustrating where the guide tip 110 on the distal end D of the shaft 102 may be configured with a set of bores 114 which retain the loop wire 104 and which bisect at a proximal end P within the shaft 102 and extend in lateral and upward direction toward the distal end D until they open on opposing lateral sides 116 of the guide tip 110. By forcing the loop wire 104 out laterally, it maintains the loop wire in its preformed shape—whether circular, horizontal-oval or otherwise—as the loop wire is extended away from the guide tip 110 or withdrawn back into the guide tip 110. In effect, the bore holes 114 “steer” the loop wire similarly to the torsion spring by forcing the lateral sides of the loop portion 106 to extend outwardly and maintain the circular shape as opposed to the oval shape of the existing devices.

As mentioned above, since the loop portion 106 may be conductive in some configurations of the snare tool, the shape and size of the loop portion 106 in this retracted configuration may also be useful for performing diathermic marking. In some configurations, the loop portion may be only partially conductive if portions of the loop wire are coated with a non-conductive material (such as Teflon®) to improve lubricity for passage of the wire 104 through the shaft and guide tip bores 114.

Another advantage of the guide tip 110 is that it is fixed to the shaft, allowing an operator to rotate the shaft and in effect rotate the loop portion 106 the same amount.

III. Wire

The wire used in the snare tool may include specific materials, structures, shapes and surface treatments to improve both the overall function of the snare tool by increasing the ability of the loop wire to grip a surface and further the efforts to maintain the loop wire in the circular shape. For the wire to be retained within the shaft 102 and also deploy outward from the distal end into a circular shape, the wire is typically made of a shape alloy such as a monofilament of a superelastic nitinol.

The wire may have a round cross-sectional shape, but in one embodiment illustrated in FIGS. 4C-4E, the shape can be modified to flatten a portion of the cross-section and create a substantially rectangular shape which would keep the loop portion flat and in a single plane in the extended position due to the inherently greater stiffness and resistance to force applied perpendicularly to the loop's plane. As illustrated in FIG. 4C, the wire loop portion 106 may be a square wire with at least one edge 107 which acts as a cutting edge. The wire loop edge 107 may be further configured to interface with an edge 109 on the wire guide 110 which acts as a second cutting edge such that both edges operate together to easily and cleanly excise a section of tissue—particularly during cold snaring. FIG. 4D is a front view illustration of the snare loop portion 106 extending from the wire guide 110 and positioned adjacent the wire guide edge 109, which may be a raised surface extending further outward than a remainder of the wire guide 110. FIG. 4E is a cross-sectional illustration taken along the C-C line in FIG. 4D showing the interface between the wire loop edge 107 and wire guide edge 109 which is utilized as the dual-cutting edges as the wire loop 106 is reduced into the wire guide 110.

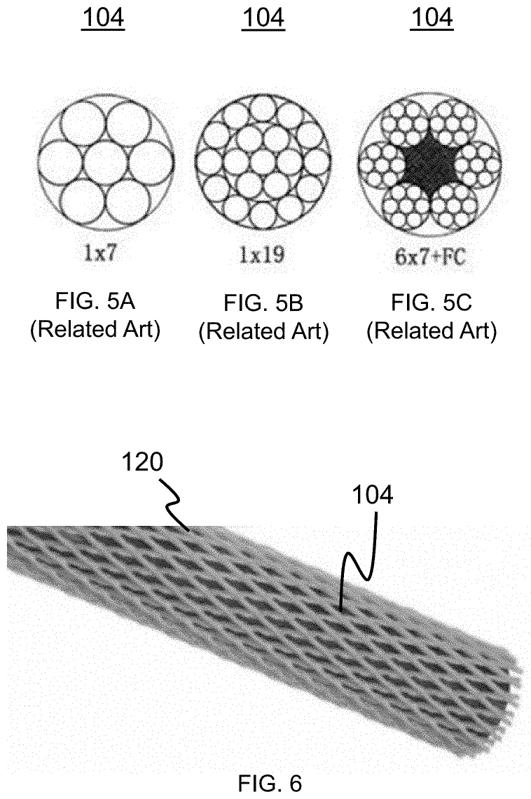

Although the embodiment above describes the use of a monofilament, in other embodiments a composite wire configuration may be utilized to increase surface texture which aids in manipulating a target tissue or surrounding tissue, especially when tissues are already difficult to grab due to the inherently slippery environment. FIG. 5A, FIG. 5B and FIG. 5C illustrate related art composite wire designs which may be utilized to increase the texture and thus the grip of the loop wire 104.

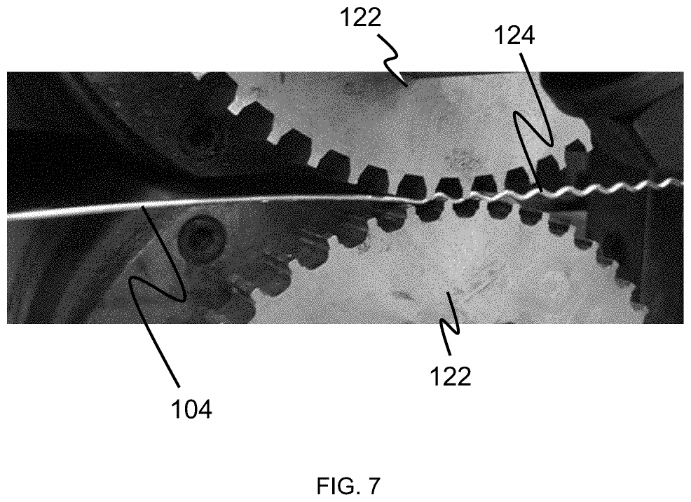

In one embodiment illustrated in FIG. 6, a braided tube 120 of fine wire covers a solid nitinol core wire 104 in order to leverage the strength and stiffness of a monofilament nitinol core but also have the added texture of an outer braided “jacket” for improving the texture of the outer surface. In another embodiment, a monofilament wire may be textured in order to enhance the grip and slip resistance. Texturing can be accomplished for example by rolling a texture into the surface of the wire (whether flat or round) or by adding a coating to the wire like an abrasive sandpaper cord, or by grit-blasting the surface of the wire to roughen it. In one embodiment illustrated in FIG. 7, the wire 104 can be crimped by pushing it through a set of gears 122 to give the wire a textured pattern 124.

IV. Rotation Mechanism

Another embodiment of the snare is illustrated in FIGS. 8A-8D. As shown in FIG. 8A, a fully-expanded snare 800A includes an outer tube 803 with a hollow shaft 802 which holds a retracted portion 804A of wire 804 with a majority forming a loop portion 804B. One end 804C of the wire 804 is anchored in a snare guide 808 disposed within a distal end 832 of the outer tube 803, while the retracted portion 804A of wire 804 is free to slide and rotate through snare guide 808 as the loop portion 804B is expanded by advancing (or reduced by pulling) the wire 804 by means of a handle mechanism (not shown) at the proximal end of outer tube 803. A coagulation bead 806 is secured near the anchored end 804C of wire 804 outside and adjacent to the distal end 832 of outer tube 803.

For insertion into an endoscope, loop portion 804B is reduced as shown in a fully-reduced configuration 800C shown in FIG. 8C. and is then expanded into the fully-expanded configuration 800A via the partially-expanded configuration in 800B for use in removing tissue, etc. In order to grab (or “snare”) tissue which needs to be examined or removed, the circular-shaped loop portion 804B in configuration 800A is reduced by pulling on wire 804A. When the loop portion 804B is fully reduced as in configuration 800C, the coagulation bead 806 is pulled into position at the distal end 832 of tube 803 to aid in marking tissue. In one embodiment, a diameter of the loop portion 804B in the fully retracted position is approximately 2 mm, such that the outside diameter of the loop portion wire 804B is small enough to then be removed from the endoscope's working channel (not shown).

In one embodiment, a consistent-shape snare described herein is capable of maintaining the loop shape as the diameter of the loop expands and contracts, making it easier to snare and remove tissue of any size. The loop may be weighted at a certain portion to maintain a desired angle which is generally preferred to be parallel with respect to a plane cutting through the base of the target tissue. In one embodiment, the consistent-shape snare is also configured to maintain the angle or alignment of the loop with respect to the catheter shaft while the loop expands and contracts (such as the half open position of FIG. 8B) so that the configurational relationship of the snare with respect to tissue being removed does not change as the size of the loop expands or contracts. In FIG. 8B, the diameter of the loop has been reduced to 13 mm, which may be considered approximately a half opening. The angle is maintained throughout the process of adjusting the size of the loop and even when pulling on the wire 804A to fully reduce the loop shape 804B to allow removal of the snare 800 from the endoscope. This reduces the risk of losing an ensnared polyp or other tissue that needs to be excised.

In one embodiment, the angle of the snare loop portion 804B may be adjustable with a moveable core wire, while in another embodiment, the snare guide 808 on the end of the outer tube 803 may help angle the snare 800 in the correct orientation to capture a polyp. The outer tube 803 of the snare may also be rotated in order to adjust the orientation of the loop 804B. Alternatively the loop 804B could be fixed in relation to the outer tube 803 and include markings on a proximal end of the outer tube 803 visible to the operator to match with the alignment of the loop 804B with respect to the distal end 832 of the outer tube 803. The outer tube 803 of the catheter may have a braid embedded to provide additional strength for the torque to be applied to the snare loop 804B.

It is desirable when using a snare loop to exert downward pressure on the circumference of the snare while it is being retracted to prevent it from slipping off an area of tissue at the same time. It is a back (most distal) portion 830 of the snare loop 804B that tends to lift and slip off while the snare is reduced (i.e., the back portion 830 flips up). Some angulation between the snare and shaft may help alleviate slippage, but in one embodiment, a flexible tent-pole configuration may be used. The pole design would spring open when the snare is opened and push the snare down, while also keeping the back end pressed down. The tent-pole configuration would need to be made of a nonconductive material.

Another embodiment would utilize guide tip on the distal end of the outer tube 803 to actuate or angle the snare loop 804B and allows adjustment of the orientation of the snare loop 804B. There is also the possibility of an additional channel that runs along the length of the scope fastened to the outer tube, through which the snare can be passed. This would allow the snare to exit more flush with the bowel wall. The working channel is offset from the rim of the endoscope, so it is always exiting at an angle relative to the bowel wall. This design helps achieve the goals of finding a way to keep the snare pressed down against the bowel wall as it closes, preventing the distal end of the snare from flipping up, and maximizing the gripping power of the snare on the mucosa.

In one embodiment, the snare loop portion 804B may be designed to adjust the angle of orientation as the loop portion 804B reduces from the expanded configuration 800A to the reduced configuration 800C in order to increase the likelihood of ensnaring the tissue of interest and maintaining the grip on the tissue as the loop portion 804B is closed and withdrawn into the outer tube 803.

FIG. 8D illustrates one configuration of the consistent-shape snare which provides for the snare wire to swivel freely to reduce torque created during operation of the snare, thus keeping the snare in the consistent-shape and angle desired during a procedure. As shown in FIG. 8D, the snare guide 808 may be configured with a housing 812 which houses the terminal point of the wire 804, but which has been fastened with a ball swivel 810 which independently rotates while connected with the wire 804 and while retaining the wire 804 within the housing 812 via retaining tabs 813. As the snare guide 808 is press-fit into the outer tube, this allows the snare wire 804 to swivel independently from the shaft 802 of the snare.

It is important to distinguish the circular shape of the opened snare (FIGS. 8A-C) versus the rounded shape of the closed snare (FIG. 8D). Note that the rounded tip can be used to deliver cautery for both diathermic marking and vessel coagulation for hemostasis or prophylactic ablation. While the concept of a ball to apply cautery (to create a well demarcated crisp ‘dot’ as opposed to an ill-defined coagulation blanch that will be difficult to distinguish from cautery artifact after snare resection) is valid, there is concern that there will be an increased amount of cautery delivered at the sphere during snare resection, potentially leading to a deeper wall burn. In one embodiment, a spherical hinged “lid” at the tip of snare which opens when the snare is extruded and closes when snare is retracted into catheter may be utilized, but the snare likely could not be pulled completely into the catheter. It will be rounded and may suffice for marking and coagulation of vessels. However, one alternative design is presented below and illustrated in FIG. 8D.

FIG. 8D illustrates how the tissue marking shape 806 may be retained in a specially-designed conical groove 814 at the end of the snare guide and/or outer tube, to allow for more accurate positioning and use of the tissue marking shape 806 with the wire 804 in a closed (retracted) position. Since the tissue marking shape 806 may be a cauterizing tool, the outer tube or snare guide 808 may be configured with an electrical contact point on the conical groove so that the tissue marking shape 806 is powered only when in the groove but is otherwise inert when the wire is extended in the circular loop and being used for other purposes. Thus, the ball would be insulated to the snare cable and only be hot when in contact with the insert inside the end of the snare tubing.

FIG. 8D illustrates an alternate rotation mechanism 816 for allowing independent rotation of the wire which is positioned further within the snare guide 808. This rotation mechanism utilizes a housing 820 which retains two ball swivels 818 retained on opposite ends thereof, with each ball swivel being connected with one of a proximal wire 804P or distal wire 804D. The distal wire 804D extends distally into the body cavity while the proximal wire 804P extends through the plug shaft 802 and out of the body cavity. Swivel loops (not shown) may be used to connect the wires 804D, 804P to the ball swivels 818, or the wires 804D and 804P may be directly connected to the swivel loops 818. This alternate rotation mechanism 816 provides independent rotation of the proximal wire within the snare guide and the distal wire in the body cavity, again preventing torque from building up and rotating the wire into an undesired position.

V. Method of Use

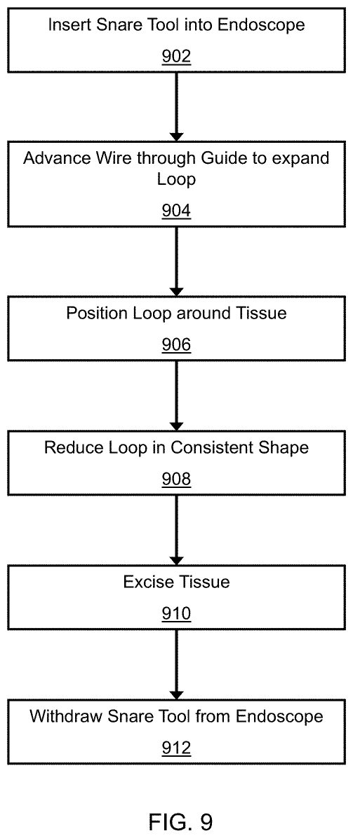

In one embodiment illustrated by the flow diagram in FIG. 9, a method of using the snare tool to excise tissue from a body cavity is provided. In step 902, a snare tool is inserted into an endoscope and into a body cavity. In step 904, a loop portion of a wire is expanded through a wire guide and into a body cavity. In step 906, the expanded loop portion is placed around a segment of tissue to be removed, after which, in step 908, the loop portion is reduced around the target tissue. If needed, in step 910, the tissue is excised using electrocautery or one or more cutting surfaces on the wire loop or guide tip. Finally in step 912, the snare tool (in its reduced configuration) is withdrawn from the endoscope.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

What is claimed is:

1. An endoscopic snare tool, comprising:
a hollow outer sheath with a proximal end and a distal end, wherein the distal end is configured to pass through a channel in an endoscope;
an adjustable wire substantially disposed within the hollow outer sheath, wherein the adjustable wire forms a circular loop portion at the distal end of the hollow outer sheath; and
wherein the circular loop portion of the adjustable wire is maintained in a substantially circular shape as a diameter of the loop shape is expanded or reduced.

2. The endoscopic snare tool of claim 1, wherein an angle of rotation of the loop shape with respect to a lengthwise axis of the hollow outer sheath may be altered.

3. The endoscopic snare tool of claim 1, wherein an angle of the loop shape with respect to the hollow outer sheath is maintained as the diameter of the loop shape is expanded or reduced.

4. The endoscopic snare tool of claim 1, further comprising a coagulation marking device disposed on a portion of the loop shape of the adjustable wire.

5. The endoscopic snare tool of claim 1, wherein the adjustable wire further comprises a torsion spring positioned at a distal tip of the circular loop portion.

6. The endoscopic snare tool of claim 5, wherein the torsion spring is located on an outer circumference of the circular loop shape.

7. The endoscopic snare tool of claim 5, wherein the torsion spring is located on an inner circumference of the circular loop shape.

8. The endoscopic snare tool of claim 1, further comprising a snare guide positioned at a distal end of the hollow outer sheath, wherein the snare guide retains a distal end of the adjustable wire with a freely rotating swivel ball connected with the adjustable wire and retained within a housing.

9. The endoscopic snare tool of claim 1, further comprising a guide tip with bisecting bores positioned on a distal end of the hollow outer sheath for guiding the circular loop shape out of the distal end.

10. The endoscopic snare tool of claim 9, wherein the bisecting bores are disposed on opposing lateral sides of the hollow outer sheath.

11. The endoscopic snare tool of claim 9, wherein the guide tip further comprises a cutting edge adjacent to a section which guides the loop shape out of the distal end.

12. The endoscopic snare tool of claim 1, wherein the adjustable wire is a monofilament formed of superelastic nitinol with a circular cross-section.

13. The endoscopic snare tool of claim 1, wherein the adjustable wire is formed with a cutting edge along an interior diameter of the circular loop portion.

14. The endoscopic snare tool of claim 1, wherein the adjustable wire is a square profile wire comprising a plurality of cutting edges along a diameter.

15. A method of ensnaring an object with an endoscopic snare tool, comprising the steps of:
inserting the snare tool through a channel in an endoscope and into a body cavity;
expanding an adjustable wire loop disposed within the snare tool into the body cavity in a substantially circular shape;
positioning the adjustable loop around the object;
reducing the adjustable loop to ensnare the object while maintaining the substantially circular shape; and
withdrawing the snare tool and reduced adjustable wire loop from the channel in the endoscope.

16. The method of claim 15, further comprising guiding the adjustable loop wire through a guide tip on opposing openings on lateral sides of the guide tip.

17. The method of claim 16, further comprising guiding the adjustable loop wire through bisecting bores within the guide tip.

18. The method of claim 15, further comprising adjusting an angle of the expanded adjustable loop wire with respect to a lengthwise axis of the channel.

19. The method of claim 15, further comprising reducing the adjustable loop to excise the object.
